(19) United States
(12) Patent Application Publication
(10) Pub. No.: US 2023/0282467 A1
(43) Pub. Date: Sep. 7, 2023

(54) STABLE ISOTOPE LABELLED INTERNAL CALIBRATORS FOR THE QUANTIFICATION OF COMPLEX MOLECULES

(71) Applicant: Micromass UK Limited, Wilmslow (GB)

(72) Inventor: Donald Cooper, Macclesfield (GB)

(73) Assignee: Micromass UK Limited, Wilmslow (GB)

(21) Appl. No.: 18/179,549

(22) Filed: Mar. 7, 2023

Related U.S. Application Data
(60) Provisional application No. 63/317,249, filed on Mar. 7, 2022.

(51) Int. Cl. H01J 49/00; G01N 30/72

(52) U.S. Cl. CPC H01J 49/0009; G01N 30/7233

(57) ABSTRACT

Provided herein are methods and systems directed to stable, isotopically labeled internal calibrators for use in mass spectrometry analysis for quantifying a target analyte in a sample. The present disclosure relates more particularly to mass spectrometry analysis where a single sample includes at least three isotopically labeled internal calibrators and the target analyte. The methods and systems described herein allows accommodation of isotope interferences arising from the use of isotopically labeled internal calibrators in quantification a target analyte. As a result, smaller quantities (e.g., lesser concentration) of isotopically labeled internal calibrators are utilized in the present technology in the generation of a calibration curve to quantify a target analyte.

### RELATED APPLICATIONS

This application claims priority to U.S. Provisional Application No. 63/317,249, filed on Mar. 7, 2022. The entire contents of the foregoing applications are hereby incorporated by reference.

### FIELD OF THE TECHNOLOGY

The present technology generally relates to isotopically labeled internal calibrators and uses thereof for quantifying one or more target analytes in a sample by mass spectrometry. The present disclosure relates more particularly to mass spectrometry analysis where a single sample includes at least three isotopically labeled internal calibrators and a target analyte. The present disclosure further relates to isotopically labeled internal calibrators that can mimic the target analyte such that at least one of the physicochemical properties of the said internal calibrators is essentially identical or similar to the corresponding physicochemical property of the target analyte, with the exception that the mass of said internal calibrators is at least 6 mass unit (amu) greater than the mass of the target analyte.

### BACKGROUND

Mass spectrometry (MS) has become invaluable across a broad range of fields and applications, including life sciences. Mass spectrometry is used for detailed mass compositional analysis of a sample, elucidating the mass-to-charge ratio of a sample constituents, from small molecules to very large proteins. In particular, liquid chromatography-mass spectrometry (LC-MS) has recently been used for quantification of drugs and biologically active compounds, mostly because of the high selectivity, sensitivity, speed, and simplicity imparted by LC/MS/MS.

The implementation of internal calibrators in quantitative MS analysis is a commonly used procedure. An internal calibrator is meant to correct for variability in dilutions, evaporation, degradation, recovery, adsorption, derivatization, and instrumental parameters such as injection volume while enabling quantification of the analytes through establishing a calibration curve. Using stable isotope labeled (SIL) calibrators, because of their similar physico-chemical characteristics to the analyte, has the additional advantage of minimizing mass detector fluctuations. Other advantages include identical optimized GC-MS, or LC-MS conditions, similar elution patterns and improved selectivity due to the mass difference.

Current approaches using SIL calibrators is directed to separating the contributions from various isotopes which usually give out partially overlapped mass spectral peaks on conventional MS systems with unit mass resolution. The empirical approaches used either ignore the contributions from neighboring isotope peaks or over-estimate them, resulting in errors for dominating isotope peaks and large biases for weak isotope peaks or even complete ignorance of the weaker peaks.

### SUMMARY

The present technology provides compounds, compositions, kits, and methods for quantifying one or more analytes in a sample by mass spectrometry without relying upon conventional calibration and its associated drawbacks and disadvantages.

Stable isotope labeled (SIL) calibrators are compounds in which several atoms in the analyte are replaced by their stable isotopes, such as ₂H (D, deuterium), ₁₃C, ₁₅N, or ₁₇O. The current approaches in the art require calibrators with at least 8-12 stable isotope labels because the current methods are directed to separating the contributions of various SIL calibrators from each other to avoid significant isotope interference. The aim of the present disclosure is to obviate or mitigate at least one disadvantage of previous methods and systems. Specifically, the present technology utilizes isotope interferences between the calibrators' signal contributions (e.g., m/z intensity) to its advantage. To illustrate, the methods disclosed herein add overlapping signal contributions of SIL calibrators together such that actual concentration of SIL calibrators (concentration of SIL calibrators spiked into a sample) needed to obtain a calibration curve decreases as compared to the concentration needed if overlapping signal contributions are ignored. Thus, one of the benefit of using the methods of the present technology is a decrease in the cost of quantitative MS analysis by reducing the actual amount of calibrators needed to obtain a calibration curve.

Any isotope interference between signals of SIL calibrators is accommodated in the methods of the present disclosure to quantify an analyte. The design of the SIL calibrators provided herein also pave the way to accommodate interferences between SIL calibrators in calibrator concentration value assignment.

In one aspect, provided herein is a composition for quantifying the amount of target analyte in a sample by mass spectrometry, comprising: at least three calibrators of known quantities, wherein the mass of at least three calibrators differs in at least 1 mass units from each other and the calibrator having the lowest mass within the at least three calibrators of known quantities have a mass at least 6 mass unit greater than the target analyte. In some embodiments, the mass of at least three calibrators differs in 1 mass units from each other. In some embodiments, the at least three calibrators of known quantities have at least one overlapping m/z peak upon their fragmentation by a mass spectrometry.

The present disclosure provides a single sample including at least three calibrators and a target analyte, wherein the mass of at least three calibrators differs in at least 1 (or 2, 3, 4, 5, . . . ) mass units. In some embodiments, the SIL calibrator having the lowest mass within the at least three calibrators have a mass at least 6 mass unit greater than the target analyte. In one embodiment, the mass of the calibrators differs in 1 mass units from each other (e.g., a first calibrator having mass M, a second calibrator having mass M+1, a third calibrator having mass M+2, a fourth calibrator having a mass M+3, . . . ). In some embodiments, the first calibrator has a mass at least 6 mass units greater than the target analyte (e.g., the target analyte having a mass M-6, the first calibrator having mass M). In some embodiments, the at least three calibrators of known quantities have at least one overlapping m/z peak upon their fragmentation by a mass spectrometry.

In some embodiments, the present disclosure provides for MS analysis where there is a single sample including a first known quantity of a first calibrator, a second known quantity of a second calibrator, a third known quantity of a third calibrator, a fourth known quantity of a fourth calibrator and the target analyte. Each calibrator and the target analyte are each distinguishable within the single sample by mass spectrometry. This avoids the need for an external calibration. Thus, by using internal calibration it is possible that an analyte is quantified by performing a single analysis of one sample so that each analysis yields a result thereby increasing the productivity and decreasing the costs per sample.

In some embodiments, the sample comprise four SIL calibrators (such as the first, second, third, or fourth internal calibrator) and the analyte. In some embodiments, the first calibrator has a mass at least 6 mass units greater than the analyte, the second calibrator has a mass at least 7 mass unit greater than the analyte, the third calibrator has a mass at least 8 mass unit greater than the analyte, and the fourth calibrator has a mass 9 mass unit greater than the analyte.

In some embodiments, the target analytes are selected from the group of immunosuppressant drugs.

In some embodiments, the target analytes are selected from tacrolimus, rapamycin, sirolimus, everolimus, and cyclosporin A.

Accordingly, in one aspect, the present technology provides calibrator compounds which can be used as internal calibrators when quantitating the amount of the target analyte in a sample. The internal calibrators include compounds which, with respect to chemical composition, structure and physicochemical properties, are similar to the analyte but which are distinguishable from the analyte based on the behavior of the internal calibrator and the analyte in a mass spectrometer. For example, the calibrators provided herein can be distinguishable from the analyte based on differences in mass and/or fragmentation pattern. The difference in mass between the calibrators and the target analyte originates from the presence of different isotopes in the calibrators relative to the analyte.

An advantage of using SIL calibrators having masses at least 1 mass unit apart from each other is that the contribution from overlapping SIL calibrators peaks is constant and predictable. Predicting the contribution from overlapping SIL calibrators allow assigning concentration values to the calibrators, which allow for a reduction in amount of calibrators needed to obtain the calibration curve. Therefore, one of the advantage of at least some of the embodiments of the present disclosure is decrease the cost of quantitative MS analysis by reducing the amount of calibrators needed to obtain a calibration curve.

A further advantage of at least some of the embodiments of the present disclosure is that the calibration calibrators are present in exactly the same matrix as the target analyte. Thus, each sample has its own perfectly matrix-matched calibration calibrators, thereby reducing or eliminating matrix effects. Another advantage of the present disclosure is reduced costs compared to conventional assays, multiplex capability, and the potential for decreasing time to result and increasing throughput, as compared to conventional methods.

Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

### DETAILED DESCRIPTION OF THE DRAWINGS

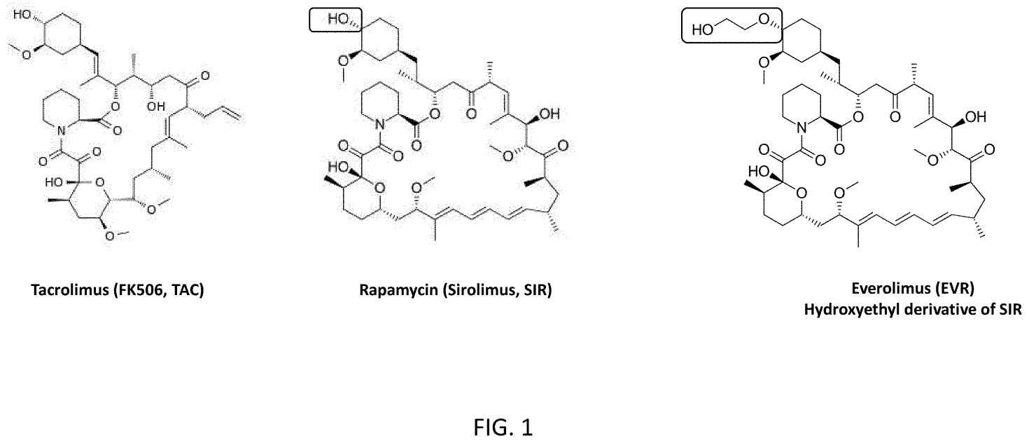

FIG. 1 illustrates exemplary analytes according to multiple embodiments of the present disclosure. Tacrolimus and Sirolimus, which are macrolide immunosuppressant drugs, are the natural products of Streptomyces species.

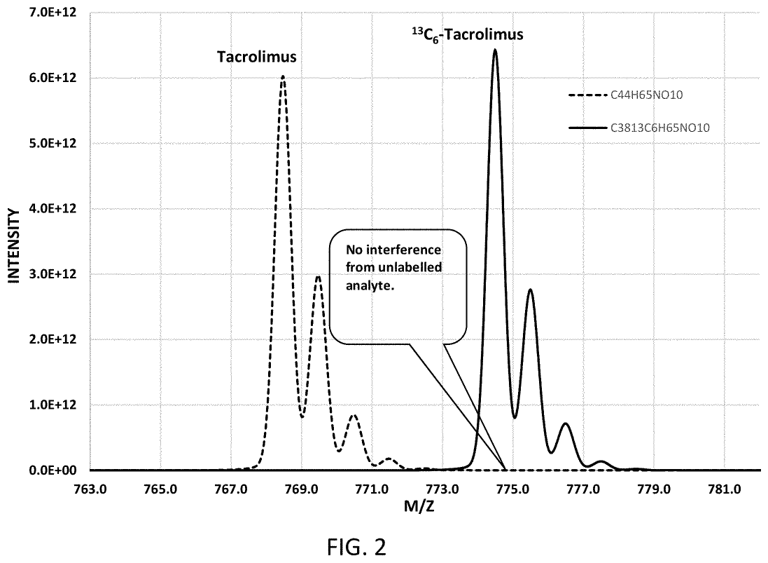

FIG. 2 shows mass spectrum of equimolar concentrations of Tacrolimus and [¹³C₆]-Tacrolimus labelled with six [¹³C₆] isotope atoms. The Y-axis on a mass spectrum is relative intensity. C₄₄H₆₅NO₁₀ and C₃₈¹³C₆H₆₅NO₁₀ represent Tacrolimus and Tacrolimus labelled with six [¹³C₆] isotope atoms, respectively.

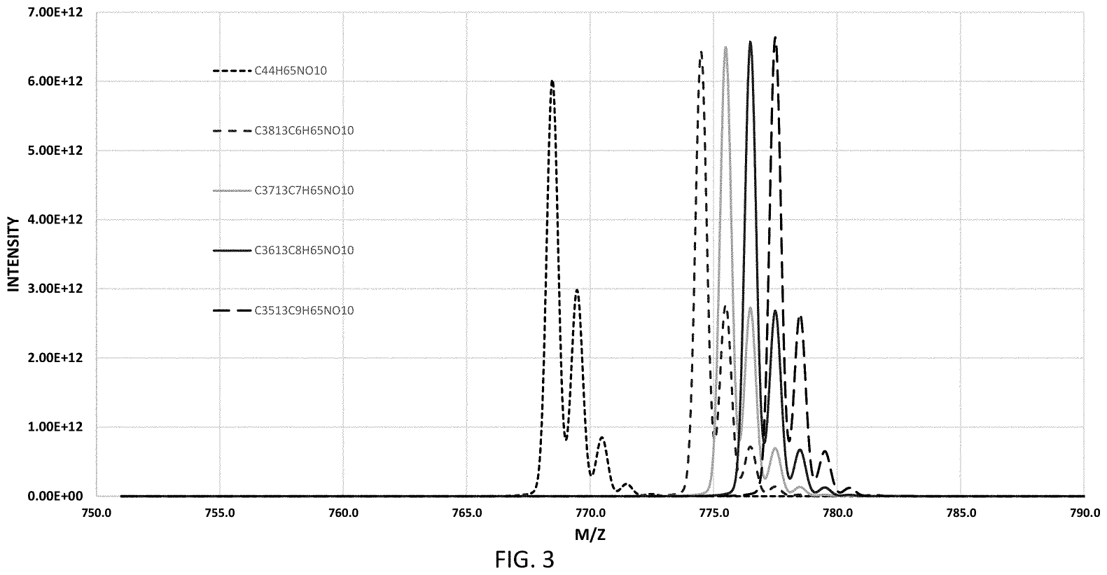

FIG. 3 shows mass spectrum of equimolar concentrations of Tacrolimus and four [¹³C₆]-Tacrolimus calibrators (SILs) labelled with six [¹³C₆] isotope atoms, with seven [¹³C₆] isotope atoms, eight [¹³C₆] isotope atoms and nine [¹³C₆] isotope atoms. The Y-axis on a mass spectrum is relative intensity. C₄H₆₅NO₁₀, C₃₈¹³C₆H₆₅NO₁₀, C₃₇¹³C₇H₆₅NO₁₀, C₃₆¹³CH₆₅NO₁₀, C₃₅¹³C₉H₆₅NO₁₀, C₃₈¹³C₆H₆₅NO₁₀ represent Tacrolimus, Tacrolimus labelled with six, seven, eight and nine [¹³C₆]isotope atoms, respectively.

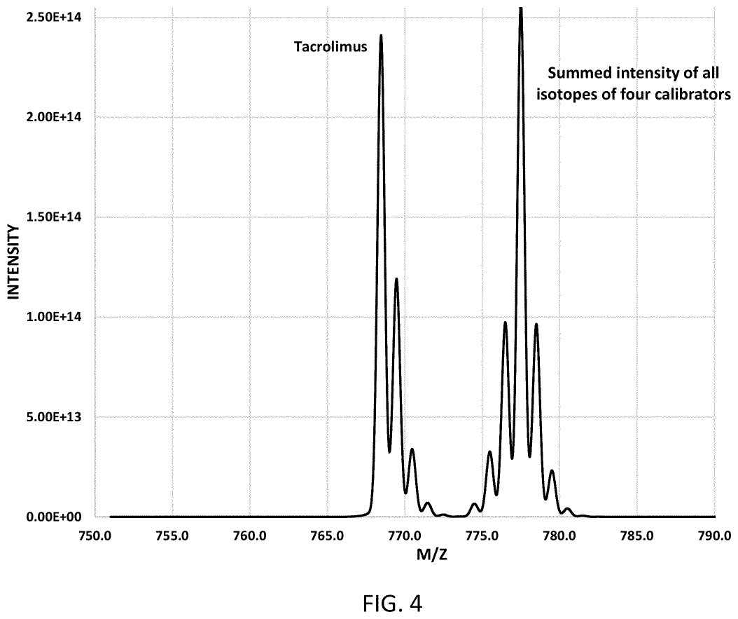

FIG. 4 shows mass spectrum of Tacrolimus and summed intensity of all four [¹³C₆]-Tacrolimus calibrators (SILs) labelled with six [¹³C₆] isotope atoms, with seven [¹³C₆] isotope atoms, with eight [¹³C₆] isotope atoms and with nine [¹³C₆] isotope atoms. The Y-axis on a mass spectrum is relative intensity.

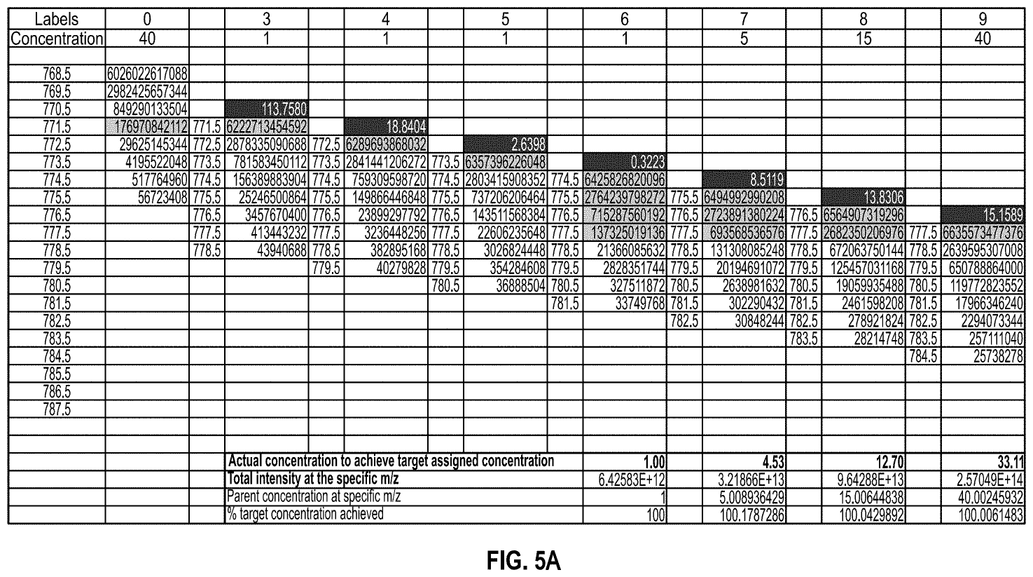

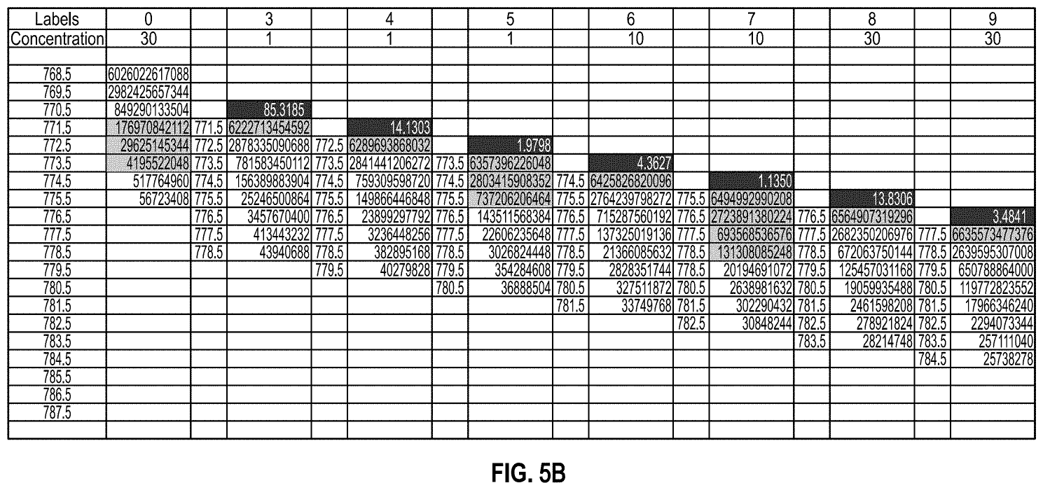

FIGS. 5A and 5B show Tacrolimus ammonium adduct isotope calculations after fragmentation (1-40 ng/mL, seven calibrators, <2% interference). FIG. 5A shows that ammonium adduct isotope calculations after fragmentation for seven [¹³C₆]-Tacrolimus calibrators (SILs) that are labelled with three [¹³C₆] isotope atoms, with four [¹³C₆] isotope atoms, with five [¹³C₆] isotope atoms, with six [¹³C₆] isotope atoms, with seven [¹³C₆] isotope atoms, with eight [¹³C₆] isotope atoms and with nine [¹³C₆] isotope atoms with corresponding concentrations of 1 ng/mL, 1 ng/mL, 1 ng/mL, 1 ng/mL, 5 ng/mL, 15 ng/mL and 40 ng/mL, respectively. Tacrolimus concentration is 40 ng/mL. FIG. 5B shows that ammonium adduct isotope calculations after fragmentation for seven [¹³C₆]-Tacrolimus calibrators (SILs) that are labelled with three [¹³C₆] isotope atoms, with four [¹³C₆] isotope atoms, with five [¹³C₆] isotope atoms, with six [¹³C₆] isotope atoms, with seven [¹³C₆]isotope atoms, with eight [¹³C₆] isotope atoms and with nine [¹³C₆] isotope atoms with corresponding concentrations of 1 ng/mL, 1 ng/mL, 1 ng/mL, 10 ng/mL, 10 ng/mL, 30 ng/mL and 30 ng/mL, respectively. Tacrolimus concentration is 30 ng/mL.

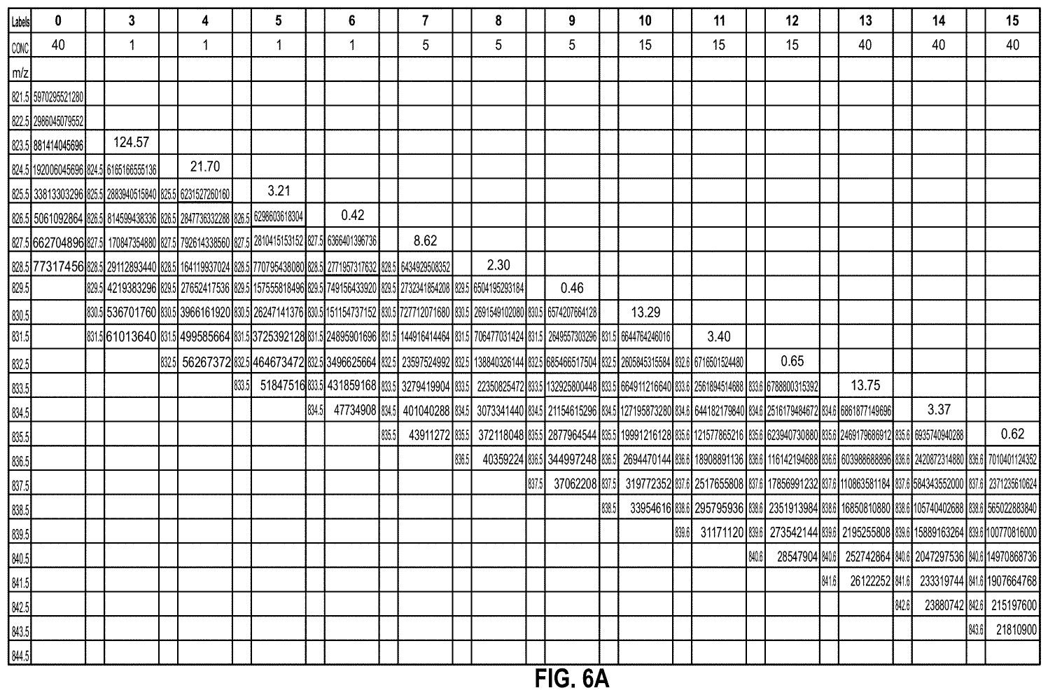

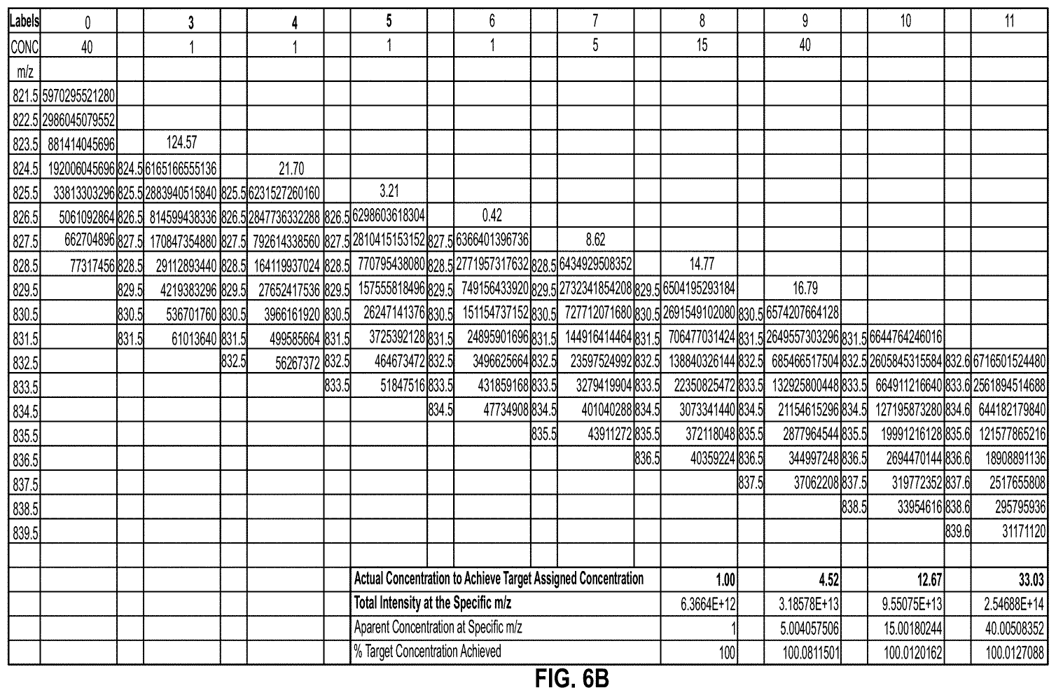

FIGS. 6A and 6B show Tacrolimus ammonium adduct isotope calculations before fragmentation (1-40 ng/mL, seven calibrators, <2% interference). FIG. 6A shows that ammonium adduct isotope calculations before fragmentation for thirteen [¹³C₆]-Tacrolimus calibrators (SILs) that are labelled with three [¹³C₆] isotope atoms, with four [¹³C₆] isotope atoms, with five [¹³C₆]isotope atoms, with six [¹³C₆] isotope atoms, with seven [¹³C₆] isotope atoms, with eight [¹³C₆]isotope atoms, with nine [¹³C₆] isotope atoms, with ten [¹³C₆] isotope atoms, with eleven [¹³C₆]isotope atoms, with twelve [¹³C₆] isotope atoms, with thirteen [¹³C₆] isotope atoms, with fourteen [¹³C₆] isotope atoms, and with fifteen [¹³C₆] isotope atoms, with corresponding concentrations of 1 ng/mL, 1 ng/mL, 1 ng/mL, 1 ng/mL, 5 ng/mL, 5 ng/mL, 5 ng/mL, 15 ng/mL, 15 ng/mL, 15 ng/mL, 40 ng/mL, 40 ng/mL, and 40 ng/mL, respectively. Tacrolimus concentration is 40 ng/mL. FIG. 6B shows that ammonium adduct isotope calculations before fragmentation for seven [¹³C₆]-Tacrolimus calibrators (SILs) that are labelled with three [¹³C₆] isotope atoms, with four [¹³C₆] isotope atoms, with five [¹³C₆] isotope atoms, with six [¹³C₆] isotope atoms, with seven [¹³C₆] isotope atoms, with eight [¹³C₆] isotope atoms, with nine [¹³C₆] isotope atoms, with ten [¹³C₆] isotope atoms, and with eleven [¹³C₆] isotope atoms with corresponding concentrations of 1 ng/mL, 1 ng/mL, 1 ng/mL, 1 ng/mL, 5 ng/mL, 15 ng/mL, 40 ng/mL, not known, not known, respectively. Tacrolimus concentration is 40 ng/mL.

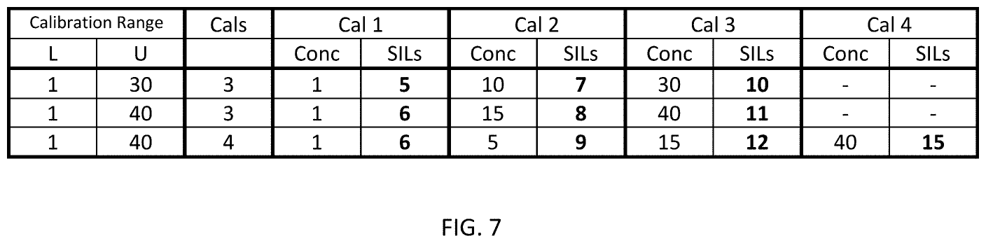

FIG. 7 shows relative concentrations of [¹³C₆]-Tacrolimus calibrators (SILs) labelled with 3-12 and 15 [¹³C₆] isotope atoms, with no more than 2% isotopic interference.

Other features and advantages of the present technology will be apparent from the following detailed description and claims.

### DETAILED DESCRIPTION

For quantification of a target analyte in a sample, it is generally necessary to first establish a calibration curve which represents the relationship between the analytical signal obtained from the particular analytical method used, e.g., peak area or peak height in MS spectra or in mass chromatograms, and the quantity of the target analyte. Thus, prior to the analysis of a sample the analytical signals of a series of calibration calibrators (e.g., the isolated target analyte in six different concentrations) have to be determined and this calibration has to be done regularly (e.g., daily). However, this procedure reduces productivity, increases the costs per sample, and moreover, renders the analysis of just one sample inefficient.

When a compound is introduced into the ion source only a portion of the total number of molecules is ionized. This portion (or ionization efficiency) depends largely on the chemical structure of the compound but, in addition, it may vary during day-to-day operation as a result of several parameters that are difficult or nearly impossible to control, such as the temperature and pressure of the ion source. Therefore, internal calibrators are considered essential in quantitative assays employing MS detection, since instrumental changes are made largely irrelevant because they affect only absolute responses, not ratios.

Quantitative detection using MS is further complicated by the effect of matrix components, for instance plasma or urine constituents. When the analyte is introduced into the ion source it will compete for ionization with other compounds introduced into the source simultaneously. Matrix components are infamous for decreasing the analyte signal, so-called ion suppression, especially in electrospray ionization (ESI)-based MS detection. The degree of ion suppression caused by matrix components may vary largely between matrices. Unfortunately, the degree of ion suppression caused by matrix components also depends on the chemical structure of the analyte. This means that if an analyte and internal calibrator are not sufficiently similar in structure, the ratio of analyte and internal calibrator detector responses may vary as a result of different degrees of ion suppression, thus compromising the quantitation. Therefore, internal calibrators in quantitative bioanalytical LC/MS assays are either structural analogues or stable isotopically labeled (SIL) analogues of the analyte. SIL internal calibrators are compounds in which several atoms in the analyte are replaced by their stable isotopes, such as ₂H (D, deuterium), ₁₃C, ₁₅N, or ₁₇O. The current state of the art requires calibrators with at least 8-12 stable isotope labels to avoid significant isotope interference. In some instances, when the analyte is a large, complex molecule, for example, a natural product of certain microorganisms, e.g., Tacrolimus, Rapamycin, Everolimus (FIG. 1), calibrators with up to 12-15 stable isotope labels would be required. Accordingly, producing the required labelled calibrators would be prohibitively costly, time-consuming, and potentially impossible to make through organic synthetic chemistry. The purpose of the present disclosure is to obviate or mitigate at least one disadvantage of previous methods and systems.

The compositions and kits of the present disclosure allows the accommodation of isotope interferences arising from the use of isotopically labeled internal calibrators in quantification of a target analyte via calibrator concentration value assignment. This leads to smaller quantities of isotopically labeled internal calibrators to be used.

### Definitions

The term “isotopes” relates to nuclides with the same number of protons but differing numbers of neutrons (i.e., they have the same atomic number and are therefore the same chemical element). Different isotopes of the same chemical element generally have essentially the same chemical characteristics and therefore behave essentially identically in chemical and/or biological systems. Therefore, for example, isotope labeled analogs of tacrolimus include compounds that are essentially identical to tacrolimus in chemical composition and structure, with the exception that at least one atom of the tacrolimus is substituted for an isotope thereof.

Isotopes relate to nuclides with the same number of protons but differing numbers of neutrons (i.e., they have the same atomic number and are therefore the same chemical element). Different isotopes of the same chemical element generally have essentially the same chemical characteristics and therefore behave essentially identically in chemical and/or biological systems. Therefore, isotope labeled analogs of a corresponding target analytes include compounds that are essentially identical to the target analyte in chemical composition and structure, with the exception that at least one atom of the target analyte is substituted for an isotope thereof.

In various embodiments, the at least one atom of the target analyte is the most abundant naturally occurring isotope and the substituted isotope of the calibrator is a less abundant isotope. For example, the target analyte can include a position with ¹H (₁₂C, ¹⁴N, ¹⁶O, or ⁸⁰Se) and the calibrator can substitute the atom in that position for ²H (¹³C, ¹⁵N, ¹⁷O, ¹⁸O, ³³S, ³⁶S, and ⁷⁴Se, respectively). The natural abundance of the isotope can be less than 49% (e.g., less than 40%, 30%, 20%, 10%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the total amount of all existing isotopes). The isotope labeled analog can use a stable isotope.

A stable isotope of an atom can be non-radioactive or radioactive. If the stable isotope is radioactive, its half-life is too long to be measured, such as a half-life longer than the age of the universe, e.g., a half-life of 13.75×10⁹ years or greater. Stable isotopes include, but are not limited to, ²H, ⁶Li, ¹¹B, ³C ¹⁵N, ¹⁷O, ¹⁸O, ²⁵Mg, ²⁶Mg, ²⁹Si, ³⁰Si, ³³S, ³⁴S, ³⁶S, ³⁷Cl, ⁴¹K, ⁴²Ca, ⁴³Ca, ⁴⁴Ca, ⁴⁶Ca, ⁴⁸Ca, ⁴⁶Ti, ⁴⁷Ti, ⁴⁹Ti, ⁵⁰Ti, ⁵⁰V, ⁵⁰Cr, ⁵³Cr, ⁵⁴Cr, ⁵⁴Fe, ⁵⁷Fe, ⁵⁸Fe, ⁶⁰Ni, ⁶¹Ni, ⁶²Ni, ⁶⁴Ni, ⁶⁵Cu, ⁶⁶Zn, ⁶⁷Zn ⁶⁸Zn, ⁷⁰Zn, ⁷¹Ga, ⁷³Ge, ⁷⁶Ge, ⁷⁴Se, ⁷⁶Se, ⁷⁷Se, ⁷⁸Se, ⁸²Se, ⁸¹Br, ⁸⁴Sr, ⁹⁶Z, ⁹⁴Mo, ⁹⁷Mo, ¹⁰⁰Mo, ⁹⁸Ru, ¹⁰²Pd, ¹⁰⁶Cd, ¹⁰⁸Cd, ¹¹³In, ¹¹²Sn, ¹¹²Sn, ¹¹⁴Sn, ¹¹⁵Sn, ¹²⁰Te, ¹²³Te, ¹³⁰Ba, ¹³²Ba, ¹³⁸La, ¹³⁶Ce, ¹³⁸Sn, ¹⁴⁸Nd, ¹⁵⁰Nd, ¹⁴⁴Sm, ¹⁵²Gd, ¹⁵⁴Gd, ¹⁵⁶Dy, ¹⁵⁸Dy, ¹⁶²Er, ¹⁶⁴Er, ¹⁶⁸Yb, ¹⁷⁰Yb, ¹⁷⁶Lu, ¹⁷⁴Hf, ^{180m1}Ta, ¹⁸⁰W, ¹⁸⁴Os, ¹⁸⁷Os, ¹⁹⁰Pt, ¹⁹²Pt, ¹⁹⁶Hg, and ²⁰⁴Pb. Examples of preferred stable isotopes include ²H, ¹¹B, ¹³C, ¹⁵N, ¹⁷O, ¹⁸O, ³³S, ³⁴S, ³⁶S, ⁷⁴Se, ⁷⁶Se, ⁷⁷Se, ⁷⁸Se, and ⁸²Se.

An isotope labeled analog can substitute between one and n atoms with isotopes, where n is the number of atoms in the target analyte molecule. In various embodiments, isotope labeled analogs can include 1, 2, 3, . . . , n substitutions, which can then form a set of internal calibrators. For example, a first calibrator can be an analog with six substitution, a second calibrator can be an analog with seven substitutions, a third calibrator can be an analog with eight substitutions, and so on. The isotope labeled analogs can vary by one or more (e.g., where more than one substitution is made between analogs and/or where the isotopes differ by more than one mass unit from the most common naturally occurring isotope) mass units. A given analog can be isotopically pure with respect to the atom in the substituted position(s).

The term “isotopically pure” can mean that at least 95% of atoms of a given type (e.g., a high abundant isotope such as ¹H) contained in a compound (such as a target analyte) have been replaced with another, preferably less abundant, isotope of the same element (e.g., ²H). For example, at least 96%, 97%, 98%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, or 99.99% or more of atoms of a given type can be replaced with another, preferably less abundant, isotope of the same element.

The term “isotopologue” refers to a species in which the chemical structure differs from a specific compound of this disclosure only in the isotopic composition thereof.

The term “calibrator” refers to a compound, material or composition used as a calibrator or control, typically to construct a calibration curve allowing to determine the unknown concentration or amount of a target analyte in a given sample. The calibrators can be used to standardize or calibrate an instrument e.g., MS spectrometry or laboratory procedure e.g., extraction, chromatographic elution.

The term “unlabeled calibrator” refers to a calibrator compound which does not possess any isotopic atom replaced by a human being.

In some embodiments, the unlabeled calibrator may refer to a target analyte. In some embodiments, the unlabeled calibrator may refer to a compound which has similar physicochemical properties to physicochemical properties of a target analyte.

The term “stable isotopically labelled calibrators,” as used herein, refers to isotopically labelled calibrators which possess stability sufficient to allow for their manufacture and which maintain the integrity of the calibrators for a sufficient period of time to be useful for the purposes detailed herein.

“D” refers to deuterium.

“¹³C” refers to carbon-13.

“¹⁵N” refers to nitrogen-15.

“¹⁸O” refers to oxygen-18.

“Replaced with carbon-13” refers to the replacement of one or more carbon atoms with a corresponding number of carbon-13 atoms.

“Replaced with nitrogen-15” refers to the replacement of one or more nitrogen atoms with a corresponding number of nitrogen-15 atoms.

“Replaced with oxygen-18” refers to the replacement of one or more oxygen atoms with a corresponding number of oxygen-18 atoms.

### Analytes

Further to the summary above, analytes or target analytes can include essentially any molecule of interest that can be detected in a mass spectrometer. The target analyte can be of interest in one or more of clinical chemistry, medicine, veterinary medicine, forensic chemistry, pharmacology, food industry, safety at work, and environmental pollution. In general, the target analyte is an organic molecule which includes at least 1 carbon atom, such as 1, 2, 3, 4, 5, 6, 7, 8, 9, 10 or more carbon atoms. The target analyte can include up to 1,000, 100, 90, 80, 70, 60, 50, 45, 40, 35, 30, 25, 20, or 15 carbon atoms. Analytes can also include inorganic analytes (e.g., phosphorous compounds, silicon compounds, inorganic polymers, and the like).

In various embodiments, target analytes of particular interest include steroids (preferably steroid hormones or sex hormones, such as testosterone, cortisol, estrone, estradiol, 17-OH-progesterone or aldosterone); immunosuppressant drugs (such as cyclosporin A, tacrolimus, sirolimus, everolimus, or mycophenolic acid); thyroid markers (such as thyroid-stimulating hormone (TSH), thyroglobulin, triiodothyronine (T3), free T3, thyroxine (T4), free T4, or ferritin); vitamins or metabolites thereof (such as the 25-hydroxy-, 1,25-dihydroxy- or 24, 25-dihydroxy-form of vitamin D2 or vitamin D3); cardiac markers (such as troponins or brain natriuretic peptide); alpha-fetoprotein; apolipoprotein, or drugs of abuse (such as hydromorphone, other opioid drugs, or therapeutic drugs).

### Samples

In general, a sample is a composition including at least one target analyte (e.g., an analyte of the class or kind disclosed above, together with a matrix). Samples can include a solid, liquid, gas, mixture, material (e.g., of intermediary consistency, such as a, extract, cell, tissue, organisms) or a combination thereof. In various embodiments, the sample is a bodily sample, an environmental sample, a food sample, a synthetic sample, an extract (e.g., obtained by separation techniques), or a combination thereof.

Bodily samples can include any sample that is derived from the body of an individual. In this context, the individual can be an animal, for example a mammal, for example a human. Other example individuals include a mouse, rat, guinea-pig, rabbit, cat, dog, goat, sheep, pig, cow, or horse. The individual can be a patient, for example, an individual suffering from a disease or being suspected of suffering from a disease. A bodily sample can be a bodily fluid or tissue, for example taken for the purpose of a scientific or medical test, such as for studying or diagnosing a disease (e.g., by detecting and/or identifying a pathogen or the presence of a biomarker). Bodily samples can also include cells, for example, pathogens or cells of the individual bodily sample (e.g., tumor cells). Such bodily samples can be obtained by known methods including tissue biopsy (e.g., punch biopsy) and by taking blood, bronchial aspirate, sputum, urine, feces, or other body fluids. Exemplary bodily samples include humor (e.g., aqueous humor and vitreous humor), whole blood, plasma, serum, umbilical cord blood (in particular, blood obtained by percutaneous umbilical cord blood sampling (PUBS), cerebrospinal fluid (CSF), saliva, amniotic fluid, breast milk, secretion, ichor, urine, feces, meconium, skin, nail, hair, umbilicus, gastric contents, placenta, bone marrow, peripheral blood lymphocytes (PBL), and solid organ tissue extract.

Environmental samples can include any sample that is derived from the environment, such as the natural environment (e.g., seas, soils, air, and flora) or the manmade environment (e.g., canals, tunnels, buildings). Such environmental samples can be used to discover, monitor, study, control, mitigate, and avoid environmental pollution. Exemplary environmental samples include water (e.g., drinking water, river water, surface water, ground water, potable water, sewage, effluent, wastewater, or leachate), soil, air, sediment, biota (e.g., soil biota), flora, fauna (e.g., fish), and earth mass (e.g., excavated material).

Food samples can include any sample that is derived from food (including beverages). Such food samples can be used for various purposes including, for example, (1) to check whether a food is safe; (2) to check whether a food contained harmful contaminants at the time the food was eaten (retained samples) or whether a food does not contain harmful contaminants; (3) to check whether a food contains only permitted additives (e.g., regulatory compliance); (4) to check whether it contains the correct levels of mandatory ingredients (e.g., whether the declarations on the label of the food are correct); or (5) to analyze the amounts of nutrients contained in the food. Exemplary food samples include edible products of animal, vegetable, or synthetic origin (e.g., milk, bread, eggs, or meat), meals, drinks, and parts thereof, such as retain samples. Food samples can also include fruits, vegetables, pulses, nuts, oil seeds, oil fruits, cereals, tea, coffee, herbal infusions, cocoa, hops, herbs, spices, sugar plants, meat, fat, kidney, liver, offal, milk, eggs, honey, fish, and beverages.

Synthetic samples can include any sample that is derived from an industrial process. The industrial process can be a biological industrial process (e.g., processes using biological material containing genetic information and capable of reproducing itself or being reproduced in a biological system, such as fermentation processes using transfected cells) or a non-biological industrial process (e.g., the chemical synthesis or degradation of a compound such as a pharmaceutical). Synthetic samples can be used to check and monitor the progress of the industrial process, to determine the yield of the desired product, and/or measure the amount of side products and/or starting materials.

### Calibrators

In one embodiment, the calibrators of the present technology may be used as stable isotopically labeled (SIL) calibrators.

In another embodiment, the calibrators disclosed herein may be used as internal calibrators in certain MS methods for quantifying a target analyte in a sample.

In some embodiments, the calibrators disclosed herein are internal calibrators. In some embodiments, internal calibrators of the present technology are stable isotopically labeled (SIL) internal calibrators.

Internal calibrators include compounds which are similar to the analyte with respect to chemical composition (e.g., empirical formula), structure (e.g., atomic arrangement and bonding), and/or physicochemical properties, but which are distinguishable from target analyte by behavior in a mass spectrometer. Exemplary calibrators disclosed herein have the same base structure as the analyte but differ slightly with respect to their molecular mass. A difference in composition and/or mass can result from replacement of an atom with a corresponding isotope of said atom (e.g., hydrogen is replaced with deuterium, carbon is replaced with carbon-13, etc.).

For example, two compounds (e.g., the internal calibrator and the analyte) can be distinguished from each other by a mass spectrometer due to differences in their mass. The masses of the two compounds (e.g., the internal calibrator and the analyte) can differ in at least 1 (or 2, 3, 4, 5, . . . ) mass units where the compounds are isotopic analogs. A difference in mass can be less than one mass unit, or a non-integer mass unit greater than one. Depending upon instrument resolution and/or a desired resolution cutoff, a difference in mass can be a difference of +0.1, 0.01, 0.001, 0.0001, 0.0001 mass units. The difference in mass between these two compounds can originate from the presence of different isotopes (e.g., low abundant isotopes in one of the two compounds vs. high abundant isotopes in the other of the two compounds) and/or different chemical moieties.

Two compounds (e.g., the first internal calibrator and the analyte) can also be distinguished from each other by a mass spectrometer due to differences in their fragmentation pattern. The fragmentation pattern of a compound relates to the compound-specific set of fragments (e.g., product/daughter ions) generated in a mass spectrometer from the compound. The two or more compounds (e.g., a calibrator and corresponding analyte, two calibrators, etc.) can fragment during the MS analysis essentially in the same way, thereby generating fragments similar in chemical composition and structure. However, in some embodiments, the fragment generated from one compound (e.g., the calibrator) can differ from the corresponding structurally similar fragment generated by the other compound (e.g., the analyte) by a difference in mass that is resolvable by the instrument being used (or by a predetermined cutoff).

The calibrators disclosed herein can mimic the target analyte such that at least one of the physicochemical properties of the internal calibrator is essentially identical to the corresponding physicochemical property of target analyte. Physicochemical properties can include any measurable property the value of which describes a physical and/or chemical state of a compound. For example, physicochemical properties include, but are not limited to, size, mass, absorbance, emission, electric charge, electric potential, isoelectric point (pi), flow rate (e.g., retention time), magnetic field, spin, solubility, viscosity, reactivity against or affinity to other substances (e.g., antibodies, enzymes), toxicity, chemical stability in a given environment, capability to undergo a certain set of transformations (e.g., molecular dissociation, chemical combination, redox reactions) under certain physical conditions in the presence of another chemical substance, polarity, and hydrophobicity/hydrophilicity. As a result, the calibrators disclosed herein are mixed well together with the target analyte in a sample solution.

In various embodiments, the calibrators disclosed herein and the target analyte are effectively indistinguishable from each other by one or more techniques commonly used to process a sample prior to mass spectrometric analysis. For example, the calibrators disclosed herein and the target analyte can be indistinguishable on the basis of solubility (in a solvent, e.g., water or an organic solvent, or a mixture of solvents), retention time (in a separation technique, such as liquid chromatography), affinity (e.g., to an antibody specific for said target analyte), dissociation constant, reactivity and/or specificity towards an enzyme (e.g., hydrolase, transferase). For example, the calibrators of the present technology can elute through a chromatography column at the same retention time as the target analyte.

Isotopes relate to nuclides with the same number of protons but differing numbers of neutrons (i.e., they have the same atomic number and are therefore the same chemical element). Different isotopes of the same chemical element generally have essentially the same chemical characteristics and therefore behave essentially identically in chemical and/or biological systems. Therefore, isotope labeled analogs of target analyte include compounds that are essentially identical to target analyte in chemical composition and structure, with the exception that at least one atom of the target analyte is substituted for an isotope thereof.

It will be recognized that some variation of natural isotopic abundance occurs in a synthesized compound depending upon the origin of chemical materials used in the synthesis. Thus, a preparation of target analyte will inherently contain small amounts of isotopologues. The concentration of naturally abundant stable hydrogen, carbon, nitrogen, and oxygen isotopes, notwithstanding this variation, is small and immaterial as compared to the degree of stable isotopic substitution of compounds of this disclosure. (See, for instance, Wada, E et al., “Natural abundance of carbon, nitrogen, and hydrogen isotope ratios in biogenic substances”, Seikagaku, 1994, 66:15; Gannes, L Z et al., “Natural abundance variations in stable isotopes and their potential uses in animal physiological ecology”, Comp Biochem Physiol Mol Integr Physiol, 1998, 119:725.

An isotope labeled analogue can replace between one and “n” atoms with isotopes, where “n” is the number of atoms in target analyte. In various embodiments, isotope labeled analogs can include 1, 2, 3, . . . , n isotopic replacements, which can then form a set of internal calibrators. Preferably, each internal calibrator contains at least 6 isotopes. Most preferably, each internal calibrator contains at least 6 carbon-13 atoms. The isotope labeled analogues can vary by one or more (e.g., where more than one substitution is made between analogs and/or where the isotopes differ by more than one mass unit from the most common naturally occurring isotope) mass units.

Internal calibrators can be selected, for example, according to the following general scheme: (a) subjecting a given target analyte to fragmentation in a mass spectrometer in order to obtain its fragmentation pattern; (b) selecting a specific fragment of said fragmentation pattern; (c) designing an isotopically labeled fragment on the basis of the fragment selected in step (b) which differs from the fragment selected in step (b) by a resolvable difference in mass and which is distinguishable from the other fragments and ions of the fragmentation pattern obtained in step (a); (d) designing an isotopically-labeled internal calibrator which will produce said isotopically labeled fragment designed in step (c) in a mass spectrometer; and (e) preparing said isotopically-labeled internal calibrator.

Internal calibrators preferably contain a sufficient number of stable isotope labels to allow them to be differentiated from target analyte using a mass spectrometer. In general, a target analyte has a characteristic isotope distribution due to the presence of low levels of naturally occurring isotopes in the molecule. Of the elements present in target analyte, carbon has the most abundant isotope in the form of carbon-13 (¹³C), which accounts for approximately 1% of all naturally occurring carbon atoms. The presence of carbon atoms in a target analyte molecule results in random occurrence of one or more ¹³C atoms, each one causing an increase in the mass of the molecule by approximately 1 Dalton. Such random occurrence of one or more 13C atoms in target analyte creates the potential for naturally occurring isotopes of target analyte to interfere internal calibrators.

In some embodiments, the SIL calibrator having the lowest mass within the at least three calibrators have a mass at least 6 mass unit greater than the target analyte. In one embodiment, the mass of the calibrators differs in 1 mass units from each other (e.g., a first calibrator having mass M, a second calibrator having mass M+1, a third calibrator having mass M+2, a fourth calibrator having a mass M+3, . . . ). In some embodiments, the first calibrator has a mass at least 6 mass units greater than the target analyte (e.g., the target analyte having a mass M-6, the first calibrator having mass M). In some embodiments, the at least three calibrators of known quantities have at least one overlapping m/z peak upon their fragmentation by a mass spectrometry.

A benefit of using SIL calibrators having masses at least 1 mass unit apart from each other is that the contribution from overlapping SIL calibrators peaks is constant and predictable. Predicting the contribution from overlapping SIL calibrators allows assigning concentration values to the calibrators, which in turn allows a reduction in the amount of calibrators needed to obtain the calibration curve. Therefore, one of the advantage of at least some of the embodiments of the present disclosure is a decrease in the cost of quantitative MS analysis by reducing the amount of calibrators needed to obtain a calibration curve.

### Compositions and Kits

Further to the summary above, a composition according to the present technology can include a first known quantity of a first calibrator and a second known quantity of a second calibrator, wherein the first known quantity and the second known quantity are different, and wherein the first calibrator, the second calibrator, and the target analyte are each distinguishable in the single sample by mass spectrometry. Kits according to the present technology can include any one or more of the compositions of the present technology, together with instructions (and/or other/additional means) for implementing the methods and/or employing the apparatuses disclosed herein.

In order to quantify a target analyte, the compositions require at least two internal calibrators corresponding to the target analyte. However, in certain circumstances, it can be advantageous to include more than two internal calibrators corresponding to the target analyte (e.g., to increase precision and/or accuracy, to decrease signal noise and/or interference or to expand the measurement range). Accordingly, a set of internal calibrators can include 2, 3, 4, 5, 6, 7, 8, 9, 10, and up to an arbitrary number of internal calibrators for a target analyte (e.g., a theoretical maximum can be determined by the maximum number of calibrators that can be designed and used for a given target analyte, for example, the number of positions that can be substituted for a stable isotope and will produce a usable signal in the contexts of the target analyte, other internal calibrators, and sample matrix). Each internal calibrator in the set should be distinguishable from each other and the target analyte by MS.

In order to quantify a target analyte, the compositions also require that at least two of the internal calibrators are present in different amounts/concentrations. In various embodiments, the amount of each internal calibrator is different. However, certain embodiments can include two or more of the internal calibrators in essentially the same amount/concentration (e.g., as long as at least two of the internal calibrators are present in different amounts/concentrations). For example, an amount of a third internal calibrator does not have to be different from the first amount of the first internal calibrator and the second amount of the second internal calibrator (e.g., the amount of the third internal calibrator can be identical to the first amount of the first internal calibrator or the second amount of the second internal calibrator; the amount of third internal calibrator cannot be identical to both the first and second internal calibrator however).

The amounts of the two or more internal calibrators can be selected to facilitate quantification of the target analyte. For example, the amounts of the internal calibrators can be selected to provide accuracy and precision over a specific analytical range of an analyte (e.g., where a specific target analyte is known to vary within a predetermined window.) In another example, the amounts of the internal calibrators can be selected to provide maximum flexibility over the analytical range of the instrument (e.g., where a target analyte is expected to vary widely or multiple analytes having different properties are to be analyzed).

In various embodiments, the two or more internal calibrators span a portion or essentially the entire analytical range of the target analyte in the sample to be analyzed. The analytical range can describe the range over which meaningful data can be collected (e.g., within pre-determined statistical parameters). The analytical range can be defined by the detection limit of an internal calibrator or target analyte in a mass spectrometer and/or the expected amount(s) of target analyte in the sample.

Thus, the amount of one or more internal calibrators can be around the expected amount of the target analyte in the sample (e.g., . . . , 50%, . . . , 95%, 96%, 97%, 98%, 99%, 100%, 101%, 102%, 103%, 104%, 105%, . . . , 150%, . . . of the expected amount of the target analyte in the sample). If the amount of the target analyte in the sample is expected to vary by orders of magnitude, then the amount of one or more internal calibrators can be, for example, . . . , 1%, . . . , 10%, . . . , 100%, . . . , 1000%, . . . , 10,000% of the expected amount of the target analyte in the sample.

The amount of one or more internal calibrators can be around/above the lower end of the analytical range of the internal calibrator in the instrument (e.g., . . . , 95%, 96%, 97%, 98%, 99%, 100%, 101%, 102%, 103%, 104%, 105%, . . . , 1000%, . . . , 10,000% of the lower end of the analytical range of the internal calibrator in the instrument). Similarly, the amount of one or more internal calibrators can be around/below the upper end of the analytical range of the internal calibrator in the instrument (e.g., 0.1%, . . . , 1%, . . . , 95%, 96%, 97%, 98%, 99%, 100%, 101%, 102%, 103%, 104%, 105%, . . . of the upper end of the analytical range of the internal calibrator in the instrument).

The relative amounts of any two internal calibrators (e.g., the internal calibrators present in the highest and lowest amounts) can be defined by a ratio, for example: 1.1, 1.15, 1.20, 1.25, 1.3, 1.4, 1.5, 2, 2.5, 3, 3.5, 4, 4.5, 5, 6, 7, 8, 9, 10, 11, 12, 13, 14, 15, 20, 25, 30, 35, 40, 45, 50, 55, 60, 65, 70, 75, 80, 90, 100, 150, 200, 250, 300, 350, 400, 450, 500, 600, 700, 800, 900, 1,000, 10,000, 100,000, 1,000,000, or more. In the embodiments including three or more internal calibrators, the differences between the amounts of internal calibrators can be linear (e.g., 2×, 3×, 4×, . . . ), exponential (e.g., 101×, 102×, 103×, . . . ), random, or a combination or variation thereof.

The present technology also encompasses compositions for quantifying more than one target analyte in a single sample. For example, a composition for quantifying a target analyte and an additional target analyte (i.e., two total analytes in a single sample) can include (i) a first known quantity of a first calibrator and a second known quantity of a second calibrator, where the first known quantity and the second known quantity are different and (ii) a third known quantity of a third calibrator and a fourth known quantity of a fourth calibrator, where the third known quantity and the fourth known quantity are different, and where the first calibrator, the second calibrator, the third calibrator, the fourth calibrator, the target analyte, and the additional target analyte are each distinguishable in the single sample by mass spectrometry. If the composition was adapted to quantify a second additional target (i.e., three total analytes in a single sample), it could further include a fifth known quantity of a fifth calibrator and a sixth known quantity of a sixth calibrator, where the fifth known quantity and the sixth known quantity are different, and where the first calibrator, the second calibrator, the third calibrator, the fourth calibrator, the fifth calibrator, the sixth calibrator, the target analyte, the additional target analyte, and the second additional target analyte are each distinguishable in the single sample by mass spectrometry.

Further compositions for quantifying multiple analytes (e.g., 2, 3, 4, 5, 6, 7, 8, 9, . . . total analytes) can be produced, for example, by combining two or more internal calibrators for each target analyte potentially present in the single sample. As described above, the two internal calibrators for each target analyte should be present in different amounts. Furthermore, in various embodiments, the target analytes and internal calibrators should all be distinguishable in the single sample by mass spectrometry. Different target analytes can each, independently, have different numbers of corresponding internal calibrators. Different internal calibrators can consist essentially of different stable isotope analogs, analogs, derivatives, metabolites, related compounds of the target analyte, or combinations thereof.

The compositions of the present technology may include dry preparations and liquid preparation (e.g., a solution, emulsion, suspension, etc.). The preparation can be determined by the requirement of compatibility with the internal calibrator (e.g., which could be incompatible with drying or unstable in liquid) or the sample (e.g., a liquid could be required to facilitate mixing and could need to be aqueous or organic or ion/pH balanced to be compatible with the sample).

Liquid preparation can include various inorganic or organic solvents, or mixtures thereof, which are compatible with the internal calibrators, sample, and MS analysis. In some embodiments, the solvent is selected for compatibility with a preparation, extraction, or separation (e.g., a chromatographic mobile phase and media). Example solvents include water, acetonitrile, aliphatic alcohols (e.g., methanol, ethanol, propanol, iso-propanol), hexafluoroacetone, and combinations thereof. The solvent can include additives, such as buffer salts (e.g., ammonium acetate), inorganic or organic acids (e.g., formic acid, trifluoroacetic acid, orthophosphoric acid, heptafluorobutyric acid), and/or inorganic or organic bases (e.g., NH₃).

Dry preparations can be prepared by various conventional drying techniques, such as, air drying, vacuum drying, spray-drying, drum drying, dielectric drying, freeze drying (e.g., lyophilization), supercritical drying, or a combination thereof. Dry preparations include preparations that are substantially free from a liquid, for example a solvent (e.g., water). In various embodiments, dry compositions can be quantified as having less than 10% w/w liquid (e.g., less than 9% w/w liquid, less than 8% w/w liquid, less than 7% w/w liquid, less than 6% w/w liquid, less than 5% w/w liquid, less than 4% w/w liquid, less than 3% w/w liquid, less than 2% w/w liquid, less than 1% w/w liquid, less than 0.5% w/w liquid, or less than 0.1% w/w liquid).

Compositions in accordance with the present technology can include one or more additional substances, e.g., substances which improve the stability of the composition, improve or facilitate the processing of a sample, and/or allow, improve or facilitate the analysis of the target analyte(s). Such additional substances include antimicrobial agents (e.g., antibiotics, azides), antioxidants, reducing agents, pH adjusting agents (e.g., inorganic and/or organic acids, bases or buffers), chelating agents (e.g., EDTA), detergents, chaotropic agents, protease inhibitors (e.g., if degradation of peptides/proteins in the sample is to be avoided), DNase inhibitors (e.g., if degradation of DNA in the sample is to be avoided), RNase inhibitors (e.g., if degradation of RNA in the sample is to be avoided), beads (e.g., beads to disrupt cell membranes or beads having ion-exchange, magnetic, size-exclusion, and/or partition properties), proteases (e.g., if degradation of peptides/proteins in the sample is desired), DNase (e.g., if degradation of DNA in the sample is desired), RNase (e.g., if degradation of RNA in the sample is desired), and solvents (e.g., if the composition is in the form of a liquid preparation).

In some embodiments, the compositions (e.g., composition used in commercial kits) include quality control (QC) material, e.g., a dry or liquid preparation containing a known amount of a target analyte, either alone or in combination with one or more internal calibrators of a set of internal calibrators which is specific for said target analyte. In various embodiments, the QC is measured in the matrix. A kit can include a pure analyte as a QC for the user to supply their own blank matrix or, alternatively, a kit can include one or more blank matrices that are pre-spiked or can be selected by the desired use to add to the pure QC material provided in the kit.

For example, a kit can include QC materials for every set of internal calibrators/target analyte. Compositions can include, for example, the internal calibrators and QC material in a single mixture. Kits can include, for example, one or more mixtures of internal calibrators as well as one or more corresponding QC materials.

Compositions in accordance with the present technology can be contained in a sample holder defining at least one sample receptacle. The sample holder can be sealable (e.g., a sealable vial, a sealable tube such as a ready-to-use tube, a sealable microtitre plate such as a 6, 24, or 96 well plate, and the like). Numerous sample receptacles, such as vials, tubes, and plates, are known in the art.

In various embodiments, compositions according to the present technology can be contained in a sample holder having one or more compartments. In one example, one or more compartments of the sample holder contain internal calibrators (i.e., one or more sets of internal calibrators as described above) in amounts that are sufficient for the analysis of one sample (e.g., including one or more target analytes) per compartment.

In some embodiments, the sample holder defines an array of sample receptacles, each receptacle containing or receiving identical compositions (i.e., sets of two or more internal calibrators for each target analyte), thereby facilitating analyzing a plurality of samples against a common analytical panel. Alternatively, a sample holder can define an array of sample receptacles, each containing or receiving different compositions (i.e., distinct sets of two or more internal calibrators for each target analyte), thereby facilitating analyzing a single sample against a plurality of analytical panels.

In another embodiment, the composition is contained in one compartment (such as a sealable tube or vial) that contains the internal calibrators (e.g., one or more sets of internal calibrators) in amounts and proportions that are sufficient for the analysis of multiple samples. The internal calibrators can be in a dry preparation, which can be reconstituted into a liquid preparation by addition of a solvent. The reconstituted liquid preparation can be added in equal aliquots to each of a plurality of samples to be analyzed, thereby ensuring that each sample includes the same quality and quantity of internal calibrators.

Compositions according to the present technology can be contained in ready-to-use reaction tubes, for example, pre-aliquoted reaction tubes that can be directly used for sample processing or analysis. Pre-aliquoted reaction tube can contain internal calibrators in amounts and proportions sufficient for the analysis of one or more samples. For example, the reaction tube may contain 3 sets of internal calibrators, wherein each set contains 4 internal calibrators and the amounts of internal calibrators within each set of internal calibrators differ from each other. The tube can be securely closed (e.g., by a screw cap, snap-on cap, or puncture cap). Example tubes can have a volume in the range of less than 1 mL, 1 to 15 mL, or 1 to 2 mL (e.g., 1.5 mL). In general, the volume of a sample receptacle can be selected on the basis of the nature and amount of sample to be processed/analyzed.

Kits according to the present technology can include any one or more of the compositions described herein, together with instructions (and/or other/additional means) for implementing the methods and/or employing the apparatuses of the present technology. Such methods and apparatuses are discussed, in turn, below.

### Methods

The present technology features methods for quantifying a target analyte by mass spectrometry. The methods include obtaining a mass spectrometer signal comprising a first calibrator signal, comprising a second calibrator signal, and potentially comprising a target analyte signal from a single sample comprising a first known quantity of a first calibrator, comprising a second known quantity of a second calibrator, and potentially comprising a target analyte. The first known quantity and the second known quantity are different, and the first calibrator, the second calibrator, and the target analyte are each distinguishable in the single sample (e.g., by mass spectrometry). The methods also include quantifying the target analyte in the single sample using the first calibrator signal, the second calibrator signal, and the target analyte signal.

In one aspect, provided herein is a method of quantifying a target analyte by mass spectrometry, the method comprising: preparing a single sample by adding at least three calibrators (e.g., 3, 4, 5, or 6) of known quantity in a single sample comprising the target analyte, wherein the mass of at least three calibrators differ in at least 1 mass units from each other and the calibrator having the lowest mass within the at least three calibrators have a mass at least 6 mass unit greater than the target analyte, wherein at least three calibrators are each different stable isotope analogs of the target analyte, wherein the target analyte is unlabeled, wherein a quantity range defined by the at least three calibrators for the target analyte spans an expected analytical range of the target analyte in the sample and wherein the amount of each calibrator differ linearly; generating a mass spectrometer signal from the single sample using a mass spectrometer comprising at least three calibrators signal, and a target analyte signal; obtaining a calibration curve, wherein the calibration curve is obtained using the at least three calibrators signal, and at least some part of at least three calibrators signal that overlaps with each other; and quantifying the target analyte using the calibration curve and the target analyte signal.

As discussed above in the context of the properties and selection of calibrators and analytes, the methods can employ more than two calibrators for a given analyte. For example, a method using three calibrators can include obtaining, from the mass spectrometer signal, a third calibrator signal from the single sample further comprising a third known quantity of a third calibrator where (i) the first known quantity, the second known quantity, and the third known quantity are different, (ii) the first calibrator, the second calibrator, the third calibrator, and the target analyte are each distinguishable in the single sample, and (iii) quantifying the target analyte includes using the third calibrator. A method using four calibrators can further include obtaining, from the mass spectrometer signal, a fourth calibrator signal from the single sample further comprising a fourth known quantity of a fourth calibrator, where (i) the first known quantity, the second known quantity, the third known quantity, and the fourth known quantity are different, (ii) the first calibrator, the second calibrator, the third calibrator, the fourth calibrator and the target analyte are each distinguishable in the single sample, and (iii) quantifying the target analyte includes using the fourth calibrator.

Additional calibrators can potentially be used to increase the precision and/or accuracy of the target analyte quantification. Additional calibrators can also be used where matrix effects are expected to obscure or distort a calibrator signal, thereby ensuring that an accurate calibration curve (or formula) can be determined despite any issues with the calibrator signals. Such additional calibrators are generally in different concentrations from the other calibrators for the given target analyte. However, in some embodiments, such additional calibrators can be in the same or essentially the same concentration as another calibrator as long as two calibrators for the given target analyte are present in different amounts.

As discussed above in the context of the properties and selection of calibrators and analytes, the methods can quantify two or more analytes in a given sample. For example, a method quantifying two analytes (e.g., a target analyte and an additional target analyte) can include (i) obtaining, from the mass spectrometer signal, a third calibrator signal, a fourth calibrator signal, and an additional target analyte signal from the single sample comprising a third known quantity of a third calibrator, comprising a fourth known quantity of a fourth calibrator, and potentially comprising an additional target analyte (where the third known quantity and the fourth known quantity are different, and where the first calibrator, the second calibrator, the third calibrator, the fourth calibrator, the target analyte, and the additional target analyte are each distinguishable in the single sample); and (ii) quantifying the additional target analyte in the single sample using the third calibrator signal, the fourth calibrator signal, and the additional target analyte signal. A method quantifying three analytes (e.g., a target analyte, additional target analyte, and second additional target analyte) can further include (i) obtaining, from the mass spectrometer signal, a fifth calibrator signal, a sixth calibrator signal, and a second additional target analyte signal from the single sample comprising a fifth known quantity of a fifth calibrator, comprising a sixth known quantity of a sixth calibrator, and potentially comprising a second additional target analyte (where the fifth known quantity and the sixth known quantity are different, and wherein the first calibrator, the second calibrator, the third calibrator, the fourth calibrator, the fifth calibrator, the sixth calibrator, the target analyte, the additional target analyte, and the second additional target analyte are each distinguishable in the single sample); and (ii) quantifying the second additional target analyte in the single sample using the fifth calibrator signal, the sixth calibrator signal, and the second additional target analyte signal.

Different methods for obtaining a mass spectrometer signal are known in the art. In various implementations, mass spectrometric analysis includes ionizing one or more compounds to generate charged molecules or molecule fragments and measuring their mass-to-charge ratios Such procedures can include the following steps: loading a mixture containing one or more compounds onto the MS instrument and vaporizing the one or more compounds; ionizing the components of the mixture, to form charged particles (ions); electromagnetically separating the ions according to their mass-to-charge ratio in an analyzer; detecting the ions (e.g., by a quantitative method); and transforming the ion signals into mass spectra.

The mass spectrometer can be operated, for example, in any of the following modes: (1) full scan, e.g., the mass spectrometer detects all ions between two distant points on the m/z scale (such as 0 and 10000); (2) Single Ion Monitoring (SIM) or Single Ion Recording (SIR), e.g., the mass spectrometer detects only ions which have a particular m/z value or which lie within a small mass m/z range (e.g., a range of 1 or 2 mass units); (3) Multiple Reaction Monitoring (MRM), e.g., in a mass spectrometer having multiple mass spectrometer units, at least two units are operated in the SIM/SIR mode.

After separation and measurement of the intensities of the ions in the mass spectrometer, mass spectra are created, for example by plotting the intensities measured for the detected ions vs. their mass-to-charge ratio (m/z). Depending on the mode by which the mass spectrometer is operated (full scan, SIM/SIR, or MRM), the mass spectra can include (1) the peaks corresponding to all ions (precursor and product ions) detected in the mass spectrometer between two distant points on the m/z scale; (2) the peaks corresponding to (a) all ions which have a particular m/z value or which lie within a very small m/z range and optionally (b) all product ions derived from the ions specified under (a); or (3) only one or more selected product/daughter ions (MRM channels).

For example, when the mass spectrometer is operated in MRM mode, one can create a single mass spectrum for a set of internal calibrators and corresponding target analyte. The single mass spectrum will contain one peak for each internal calibrator and, if present in the sample, one peak for the corresponding target analyte. Alternatively, multiple mass spectra can be created for the first set of internal calibrators and corresponding target analyte, where each of the multiple mass spectra only represents one of the internal calibrators or corresponding target analyte. Such single mass spectrum or multiple mass spectra can be created for each set of internal calibrators and corresponding target analyte.

Mass spectra created using MRM channels and where peak intensities are plotted against time (such as retention time if the mass spectrometer is coupled to a SPE, chromatography, or electrophoresis device) are often described as mass chromatograms. Thus, the term mass spectra, as used herein, can also relate to mass chromatograms (e.g., where the MS operates in MRM mode).

Next, the MS signal intensities (or relative signal intensities) of the ions representative of each of the internal calibrators and corresponding target analyte(s) are determined. The signal intensities of the ions in the mass spectra (e.g., the intensities of the peaks corresponding to these ions) can be determined on the basis of the peak height or peak area, for example on the basis of peak area such as by integrating the signal intensity of a specific ion with respect to time. The intensities of the ion signals in the mass spectrum/spectra can be normalized e.g., to 100%, to the most intense ion signal detected.

The target analyte in the single sample may be quantified using the first calibrator signal, the second calibrator signal, and the target analyte signal. The methods include quantifying the target analyte using the target analyte signal and a calibration curve or algebraic equation (i.e., based upon the calibrator signals). For example, the method can include (i) obtaining a calibration curve from the first calibrator signal and the second calibrator signal; and (ii) quantifying the target analyte using the calibration curve and the target analyte signal. Alternatively, the method can include quantifying the target analyte algebraically using the first calibrator signal, the second calibrator signal, and the target analyte signal. In various embodiments (e.g., two or more calibrators for a given target analyte, two or more target analytes, and combinations thereof), the quantifying step can be carried out manually (e.g., using pencil and paper, a calculator, or a spreadsheet, for example in a one-off, research, or development setting) or automatically (e.g., using a programmed machine or purpose-built machine, for example in a high-throughput or commercial setting).

Calibration curves can be obtained by applying a suitable regression algorithm (e.g., a Gauss least-square fitting method) to the data. Suitable regression algorithms can include the following steps: (1) selecting a mathematical function (model); (2) fitting the function from the experimental data; and (3) validating the model. The function can be, but is not necessarily, linear over the entire analytical range. Where the method is quantifying multiple target analytes, the step of creating a calibration curve using the corresponding calibrator signals can be performed for each set of internal calibrators, thereby creating a distinct calibration curve for each corresponding target analyte.

As discussed above, the compositions, the kits, and the methods of the present disclosure can be used for quantifying a target analyte by mass spectrometry. The mass spectrometer can include an ion source such as an Electrospray ionization (“ESI”) ion source; an Atmospheric Pressure Photo Ionization (“APPI”) ion source; an Atmospheric Pressure Chemical Ionization (“APCI”) ion source; a Matrix Assisted Laser Desorption Ionization (“MALDI”) ion source; a Laser Desorption Ionization (“LDI”) ion source; an Atmospheric Pressure Ionization (“API”) ion source; a Desorption Ionization on Silicon (“DIOS”) ion source; an Electron Impact (“EI”) ion source; a Chemical Ionization (“CI”) ion source; a Field Ionization (“FI”) ion source; a Field Desorption (“FD”) ion source; an Inductively Coupled Plasma (“ICP”) ion source; a Fast Atom Bombardment (“FAB”) ion source; a Liquid Secondary Ion Mass Spectrometry (“LSIMS”) ion source; a Desorption Electrospray Ionization (“DESI”) ion source; a Nickel-63 radioactive ion source; an Atmospheric Pressure Matrix Assisted Laser Desorption Ionization ion source; and a Thermospray ion source.

The mass spectrometer can include a mass analyzer such as a quadrupole mass analyzer; a 2D or linear quadrupole mass analyzer; a 3D quadrupole mass analyzer; a 2D or linear quadrupole ion trap mass analyzer; a 3D quadrupole ion trap mass analyzer; a Penning trap mass analyzer; an ion trap mass analyzer; a magnetic sector mass analyzer; Ion Cyclotron Resonance (“ICR”) mass analyzer; a Fourier Transform Ion Cyclotron Resonance (“FTICR”) mass analyzer; an electrostatic or orbitrap mass analyzer; a Fourier Transform electrostatic or orbitrap mass analyzer; a Fourier Transform mass analyzer; a Time of Flight mass analyzer; an orthogonal acceleration Time of Flight mass analyzer; and a linear acceleration Time of Flight mass analyzer. The mass spectrometer can include an ion mobility analyzer.

The mass spectrometer can include an ionization sources such as an Electrospray ionization (“ESI”) ion source; an Atmospheric Pressure Photo Ionization (“APPI”) ion source; an Atmospheric Pressure Chemical Ionization (“APCI”) ion source; a Matrix Assisted Laser Desorption Ionization (“MALDI”) ion source; a Laser Desorption Ionization (“LDI”) ion source; an Atmospheric Pressure Ionization (“API”) ion source; a Desorption Ionization on Silicon (“DIOS”) ion source; an Electron Impact (“EI”) ion source; a Chemical Ionization (“CI”) ion source; a Field Ionization (“FI”) ion source; a Field Desorption (“FD”) ion source; an Inductively Coupled Plasma (“ICP”) ion source; a Fast Atom Bombardment (“FAB”) ion source; a Liquid Secondary Ion Mass Spectrometry (“LSIMS”) ion source; a Desorption Electrospray Ionization (“DESI”) ion source; a Nickel-63 radioactive ion source; an Atmospheric Pressure Matrix Assisted Laser Desorption Ionization ion source; and a Thermospray ion source.

### Examples

Unless indicated otherwise, all techniques, including the use of kits and reagents, were carried out according to the manufacturers' information, methods known in the art, or as described, for example, in Tietz Text Book of Clinical Chemistry 3 Edition (Burtis, C. A. & Ashwood, M. D., Eds.) W. B. Saunders Company, 1999; Guidance for Industry. Bioanalytical Method Validation. USA: Centre for Drug Evaluation and Research, US Department of Health and Social Services, Food and Drug Administration, 2001; and Sambrook et al., Molecular Cloning: A Laboratory Manual, 2nd Edition (1989) Cold Spring Harbor Laboratory Press, Cold Spring Harbor, N.Y. The methods used below and described in these references are hereby incorporated by reference in their entirety.

### Consideration of the Minimum Number of Stable Isotope Labels Required in the Design of a Set of Internal Tacrolimus Calibrators

Of those elements present in Tacrolimus (C₄₄H₆₅NO₁₀), a macrolide immunosuppressant drug, carbon has the most abundant isotope in the form of carbon-13 (¹³C) which accounts for approximately 1% of all naturally occurring carbon atoms. The presence of ten carbon atoms in a Tacrolimus molecule provides the opportunity for the random occurrence of one or more ¹³C atoms, each one causing an increase in the mass of the molecule by approximately 1 Dalton. As a result, Tacrolimus has a characteristic isotope distribution, i.e., characteristic series of peaks with different intensities (FIG. 2).

The internal calibrators of the present technology (e.g., at least three known quantity of calibrators) contain sufficient number of stable isotope labels (e.g., at least six carbon-13 atoms) to allow them to be differentiated from target analyte (FIG. 2).

The internal calibrator (i.e., six stable isotopes, i.e., ¹³C labeled analogs of Tacrolimus) and the analyte can be distinguished from each other on the basis of their MS characteristics such that in a single analysis, the responses for the analogs and the analyte can be measured simultaneously. This allows an individual calibration curve to be constructed and a result generated for each sample from a single analysis of a single sample (FIG. 2). FIG. 2 shows that in order to avoid any overlapping peaks between Tacrolimus and an internal calibrator, minimum six carbon atoms of Tacrolimus needs to be replaced by an stable isotope, i.e., ¹³C. FIG. 2 provides insight for determining the theoretical minimum number of stable isotope labels required as a starting point to design a set of internal calibrators that can be used to quantify the amount of an analyte, e.g., a macrolide immunosuppressant drugs, in a sample. Factors to consider include, for example, (1) the isotope distribution of analyte, (2) the dynamic range of the assay, and (3) the maximum allowable error in the result of the assay.

### The Analysis of Tacrolimus Using Multiple Internal Calibrators in a Single Analysis

In some examples, the internal calibrators of the present technology differ in at least 1 mass units from each other. Due to random occurrence of one or more ¹³C atoms, the calibrators differ in at least 1 (or 2, 3, 4, 5, . . . ) mass units apart from each other may have at least one overlapping m/z peaks upon their fragmentation as shown in FIG. 3. The sample presented in FIG. 3 includes four SIL calibrators (i.e., the first, second, third, or fourth internal calibrator), i.e., [¹³C₆]-Tacrolimus, labelled with ¹³C atoms and the analyte (Tacrolimus). The first calibrator (+6) has a mass at least 6 mass units greater than Tacrolimus, the second calibrator (+7) has a mass at least 7 mass unit greater than Tacrolimus, the third calibrator (+8) has a mass at least 8 mass unit greater than Tacrolimus, and the fourth calibrator (+9) has a mass 9 mass unit greater than Tacrolimus.

The methods for quantification of target analytes disclosed herein benefits from the overlapping m/z peaks of the calibrators to obtain a calibration curve. Since the methods disclosed herein add overlapping signal contributions of SIL calibrators together, actual concentration of SIL calibrators (concentration of calibrators spiked into a sample) needed to obtain a calibration curve decreases. In other words, since overlapping signal intensities of SIL calibrators are summed together as shown in FIG. 4, the actual concentration of each SIL calibrator needed to achieve a target concentration at specific m/z values to obtain a calibration curve will be less. The method presented herein, therefore, provides a great advantage of using lesser amount of calibrators compared to conventional SIL calibrators methods.

Tacrolimus Ammonium Adduct Isotope Calculations (1-40 ng/mL, Four SIL Calibrators Labelled with (6, 7, 8 and 9) [¹³C] Atoms, <2% Interference)

Tacrolimus does not form an intense fragment in MS/MS. Therefore, Tacrolimus is analyzed using a mobile phase that contains ammonium acetate or ammonium formate so that in electrospray positive ionization mode, it can form an ammonium adduct (m/z 821.5). Fragmentation causes the loss of the ammonium adduct and two water molecules (m/z 821.5>m/z 768.5) and gives a relatively high signal. FIGS. 5A and 5B show MS signals of various SIL calibrators after fragmentation (m/z 768.5); FIGS. 6A and 6B show MS signals of various SIL calibrators before fragmentation (m/z 821.5).

FIGS. 5A and 5B show overlapping MS signal between four SIL calibrators labelled with (6, 7, 8 and 9) [¹³C] atoms. FIG. 5A also shows calibrators labeled with 3, 4 and 5 [¹³C] atoms; and FIG. 5B also provides MS signal data for SIL calibrators labelled with 3, 4 and 5 [¹³C] atoms. The calculations obtained using the methods presented herein shows that the actual concentration of each SIL calibrator needed to achieve a target concentration at specific m/z values to obtain a calibration curve will be less. That is, the amount of each SIL calibrator that is spiked into a sample will be less compared to the amount of each SIL calibrator used in a method that does not accommodate the overlapping signals between calibrators. For example, due to signal contributions of other SIL calibrators, the actual concentration of the fourth calibrator (+9) needed to be added into a sample is 33 ng/mL in order to obtain target (apparent) concentration of 40 ng/mL at specific m/z value. Using lesser quantities of SIL calibrators brings an advantage of greatly reducing the cost of MS analysis for complex molecules (e.g., macrolide immunosuppressant drugs). FIGS. 6A and 6B show overlapping MS signal between four SIL calibrators labelled with (6, 7, 8 and 9) [¹³C] atoms before fragmentation. FIG. 6A also shows calibrators labeled with 3, 4, 5, 10, 11, 12, 13, 14, 15 and 16 [¹³C] atoms; and FIG. 6B also provides MS signal data for SIL calibrators labelled with 3, 4, 5, 10 and 11 [¹³C] atoms. Similar to FIGS. 5A and 5B, the amount of each SIL calibrator that is spiked into a sample will be less compared to the amount of each SIL calibrator used in a method that does not accommodate the overlapping signals between calibrators.

In order to properly obtain a calibration curve and quantitively analyze Tacrolimus in a sample, the relative concentrations of multiple SIL calibrators labeled with (3, 4, 5, 6, 7, 8, 9, 10, 11, 12 and 15) [¹³C] atoms were investigated. FIG. 7 shows that when four SIL calibrators labelled with (6, 7, 8 and 9) [¹³C] atoms used in the sample, lesser concentrations of the calibrators are needed compared to other calibrators, which provides a great advantage for routine analysis of drugs reducing the cost of the analysis.

We claim:

1. A composition for quantifying the amount of target analyte in a sample by mass spectrometry, comprising:
at least three calibrators of known quantities, wherein the mass of at least three calibrators differ in at least 1 mass units from each other and the calibrator having the lowest mass within the at least three calibrators of known quantities have a mass at least 6 mass unit greater than the target analyte.

2. The composition of claim 1, wherein the mass of at least three calibrators differs in 1 mass units from each other.

3. The composition of claim 1, wherein the at least three calibrators of known quantities have at least one overlapping m/z peak upon their fragmentation by a mass spectrometry.

4. The composition of claim 1, wherein the target analytes are selected from tacrolimus, rapamycin, sirolimus, everolimus, and cyclosporin A.

5. The composition of claim 1, wherein at least three calibrators are each different stable isotope analogs of the target analyte.

6. A kit for quantifying the amount of target analyte in a sample by mass spectrometry, the kit comprising:
(i) the composition of claim 1;
(ii) instructions for (a) obtaining a mass spectrometer signal comprising at least three calibrators signal, and a target analyte signal from a single sample comprising at least three calibrators of known quantities, and the target analyte, and (b) quantifying the amount of target analyte in the single sample using the at least three calibrators signal, the target analyte signal, and at least some part of at least three calibrators signal that overlaps with each other.

7. A method of quantifying a target analyte by mass spectrometry, the method comprising:
preparing a single sample by adding at least three calibrators of known quantities in a single sample comprising the target analyte,
wherein the mass of at least three calibrators differs in at least 1 mass units from each other and the calibrator having the lowest mass within the at least three calibrators have a mass at least 6 mass unit greater than the target analyte,
wherein at least three calibrators are each different stable isotope analogs of the target analyte,
wherein the target analyte is unlabeled,
wherein a quantity range defined by the at least three calibrators for the target analyte spans an expected analytical range of the target analyte in the sample and wherein the amount of each calibrator differs linearly;
generating a mass spectrometer signal from the single sample using a mass spectrometer comprising at least three calibrators signal, and a target analyte signal;
obtaining a calibration curve, wherein the calibration curve is obtained using the at least three calibrators signal, and at least some part of at least three calibrators signal that overlaps with each other; and quantifying the target analyte using the calibration curve and the target analyte signal.
